(19) United States
(12) Patent Application Publication
(10) Pub. No.: US 2023/0409895 A1
(43) Pub. Date: Dec. 21, 2023

(54) ELECTRON ENERGY ESTIMATION MACHINE LEARNING MODEL

(71) Applicant: Microsoft Technology Licensing, LLC, Redmond, WA (US)

(72) Inventors: Hongbin LIU, Redmond, WA (US); Guang Hao LOW, Redmond, WA (US); Matthias TROYER, Clyde Hill, WA (US); Chi CHEN, Bellevue, WA (US)

(73) Assignee: Microsoft Technology Licensing, LLC, Redmond, WA (US)

(21) Appl. No.: 17/806,705

(22) Filed: Jun. 13, 2022

(51) Int. Cl. G06N 3/08; G06N 10/60

(52) U.S. Cl. CPC G06N 3/08; G06N 10/60

(57) ABSTRACT

A computing system including one or more processing devices configured to generate a training data set. Generating the training data set may include generating training molecular structures, respective training Hamiltonians, and training energy terms. Computing the training energy terms may include, for each of the training Hamiltonians, computing a kinetic energy term, a nuclear potential energy term, an electron repulsion energy term, and an exchange energy term using Hartree-Fock (HF) estimation. Computing the training energy terms may further include, for a first subset of the training Hamiltonians, computing dynamical correlation energy terms using coupled cluster estimation. Computing the training energy terms may further include, for a second subset of the first subset, generating truncated Hamiltonians and computing static correlation energy terms using complete active space (CAS) estimation. The one or more processing devices may train an electron energy estimation machine learning model using the training data set.

### BACKGROUND

Computing the total energy of the electrons in a molecule is one of the most fundamental problems in computational chemistry. The total energy of the electrons may, for example, be used to determine the stability and reactivity of the molecule. Accordingly, estimates of the total energy of the electrons in a molecule may be used when creating simulations of chemical reactions, predicting the properties of newly designed compounds, and designing chemical manufacturing processes.

The total energy of the electrons in a molecule may be computed by solving the Schrödinger equation for a wavefunction of the electrons included in the molecule. The total energy is computed as an eigenvalue of a Hamiltonian included in the Schrödinger equation. However, computing an exact solution to the Schrödinger equation is an exponentially scaling problem as a function of the number of electrons. Accordingly, methods of computing approximate solutions to the Schrödinger equation for the electrons included in molecules have been developed.

### SUMMARY

According to one aspect of the present disclosure, a computing system is provided, including one or more processing devices configured to generate a training data set. Generating the training data set may include generating a plurality of training molecular structures and computing a respective plurality of training Hamiltonians of the training molecular structures. Based at least in part on the plurality of training Hamiltonians, generating the training data set may further include computing a plurality of training energy terms associated with the training molecular structures. Computing the plurality of training energy terms may include, for each of the training Hamiltonians, computing respective estimated values of a kinetic energy term, a nuclear potential energy term, an electron repulsion energy term, and an exchange energy term using Hartree-Fock (HF) estimation. Computing the plurality of training energy terms may further include, for each training Hamiltonian included in a first proper subset of the plurality of training Hamiltonians, computing a respective dynamical correlation energy term using coupled cluster estimation. Computing the plurality of training energy terms may further include, for each training Hamiltonian included in a second proper subset of the first proper subset, generating a truncated Hamiltonian for the training molecular structure, and based at least in part on the truncated Hamiltonian, computing a respective static correlation energy term using complete active space (CAS) estimation. The one or more processing devices may be further configured to train an electron energy estimation machine learning model using the plurality of training molecular structures and the plurality of training energy terms included in the training data set.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 schematically shows a computing system during training of an electron energy estimation machine learning model, according to one example embodiment.

FIG. 2 schematically shows the example computing system of FIG. 1 in additional detail when training inputs included in a training data set are generated.

FIG. 3 schematically shows the computing system of FIG. 1 the plurality of training energy terms included in the training data set are generated.

FIG. 4 schematically shows the computation of a static correlation energy term in additional detail, according to the example of FIG. 1.

FIG. 5 schematically shows a first training phase, a second training phase, and a third training phase in which the electron energy estimation machine learning model may be trained, according to the example of FIG. 1.

FIG. 6A shows a plurality of conformers generated for a stable molecule, according to the example of FIG. 1.

FIG. 6B shows a plurality of perturbations of a conformer generated for the stable molecule of FIG. 6A.

FIG. 7 schematically shows the computing system during runtime when inferencing is performed at the electron energy estimation machine learning model, according to the example of FIG. 1.

FIG. 8A shows a flowchart of a method for use with a computing system to train an electron energy estimation machine learning model, according to the example of FIG. 1.

FIG. 8B shows additional steps of the method of FIG. 8A that may be performed in some examples when the plurality of training molecular structures are generated.

FIG. 8C shows additional steps of the method of FIG. 8A that may be performed in some examples when generating the training data set.

FIG. 8D shows additional steps of the method of FIG. 8A that may be performed in some examples when training the electron energy estimation machine learning model.

FIG. 8E shows additional steps of the method of FIG. 8A that may be performed during runtime in some examples.

FIG. 9 shows a schematic view of an example computing environment in which the computing system of FIG. 1 may be instantiated.

### DETAILED DESCRIPTION

A variety of different methods of have been developed for estimating the total electronic energy of a molecule. When selecting an estimation method, there is a tradeoff between accuracy and cost (e.g. in terms of processing time or memory use). For example, an approximate method of estimating the total electronic energy may exclude some interactions from consideration. When the total electronic energy of a molecule is estimated, the total electronic energy may be expressed as a sum of a plurality of terms:

Eₜₒₜₐₗ=E_{kinetic}+Eₚₒₜₑₙₜᵢₐₗ+E_{Coulomb}+E_{exchange}+E_{correlation-d}+E_{correlation-s}

The different terms in the above equation account for different proportions of the total energy and have different levels of computational complexity. For example, the first four terms of the above equation typically account for over 95% of the total energy and can be computed exactly with O(N⁴) complexity, where N is the number of electrons. The dynamical correlation energy term E_{correlation-d} typically contributes less than 5% of the total energy and may be accurately approximated at O(N^{6˜7}) scaling. The static correlation energy term E_{correlation-s} typically contributes less than 1% of the total energy, but exact computation of the static correlation energy term E_{correlation-s} is an exponentially scaling problem. This exponential scaling presents a challenge when simulating the behavior of molecules in reactions where 1% of the total energy is a relevant amount, such as many catalysis reactions.

Machine learning models have previously been developed to approximate the total electronic energies of molecules, including the static correlation energy term. However, such existing models typically have low accuracy when estimating the total electronic energies of molecules that have significant static correlation energy. In addition, such existing models typically have low transferability. The above deficiencies of existing machine learning models used for total electronic energy estimation typically occur as a result of having only small amounts of training data that include the static correlation energy. Since, using existing techniques, the static correlation energy is impractical to compute except for very small molecules, the training data sets of such existing machine learning models have not included large, representative samples of static correlation energy data.

In order to overcome the above challenges in total electronic energy approximation, the systems and methods discussed below are provided. These systems and methods may allow for efficient generation of a training data set that includes larger quantities of static correlation energy data than have been used to train previous machine learning models for estimating the total electronic energy of molecules. Using the systems and methods discussed below, a machine learning model may be trained using this training data set, and inferencing may be performed at the trained machine learning model. Thus, the total electronic energies of molecules may be accurately predicted at the trained machine learning model.

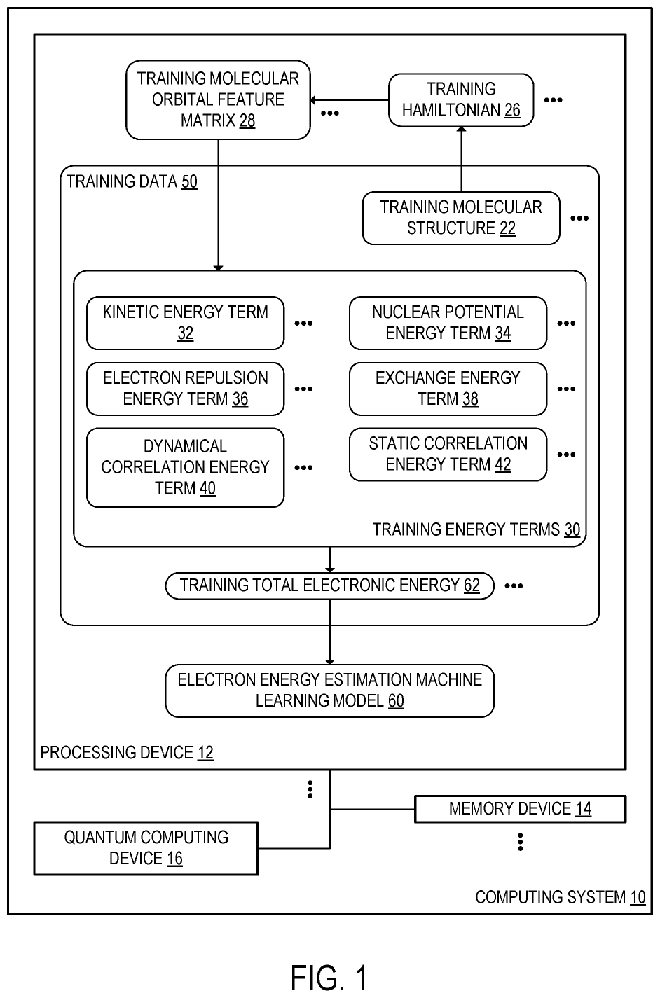

FIG. 1 schematically shows a computing system 10, according to one example embodiment. FIG. 1 shows the computing system 10 when an electron energy estimation machine learning model 60 is trained using a training data set 50, as discussed in further detail below. The computing system 10 may include one or more processing devices 12 that are configured to execute instructions to perform computing processes. The one or more processing devices 12 may, for example, include one or more central processing units (CPUs), graphical processing units (GPUs), application-specific integrated circuits (ASICs), field-programmable gate arrays (FPGAs), specialized hardware accelerators, or other types of processing devices. The computing system 10 may further include one or more memory devices 14 that are communicatively coupled to the one or more processing devices 12. The one or more memory devices 14 may, for example, include one or more volatile memory devices and/or one or more non-volatile memory devices.

The computing system 10 may be instantiated in a single physical computing device or in a plurality of communicatively coupled physical computing devices. For example, at least a portion of the computing system 10 may be provided as a server computing device located at a data center. In such examples, the computing system 10 may further include one or more client computing devices configured to communicate with the one or more server computing devices over a network.

In some examples, the computing system 10 may include a quantum computing device 16 among the one or more processing devices 12. The quantum computing device 16 may have a quantum state that encodes a plurality of qubits. When quantum computation is performed at the quantum computing device 16, measurements may be performed on the quantum state to apply logic gates to the plurality of qubits. The results of one or more of the measurements may be output to other portions of the computing system 10 as results of the quantum computation. The quantum computing device 16 may be configured to communicate with one or more other processing devices 12 of the one or more processing devices 12, and/or with the one or more memory devices 14.

The one or more processing devices 12 included in the computing system 10 may be configured to generate a training data set 50 for the electron energy estimation machine learning model 60. Generating the training data set 50 may include generating a plurality of training molecular structures 22. Each of the training molecular structures 22 may include respective indications of a plurality of atoms and one or more bonds between the atoms. The locations of the atoms may be expressed in three-dimensional coordinates.

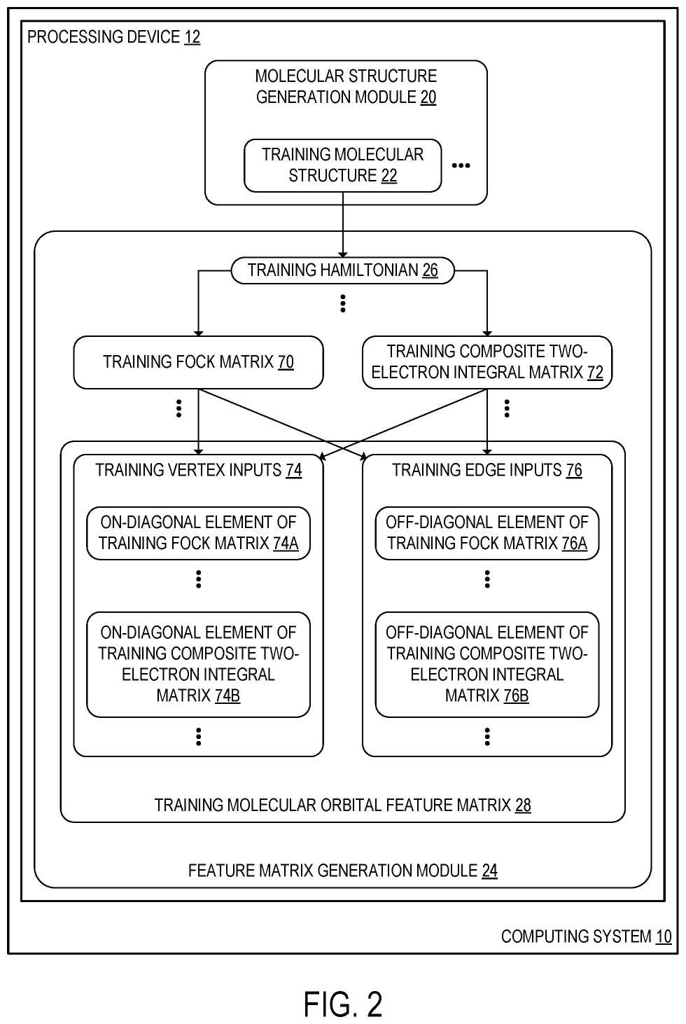

FIG. 2 schematically shows the computing system 10 of FIG. 1 in additional detail when training inputs included in the training data set 50 are generated, according to one example. As shown in the example of FIG. 2, the one or more processing devices 12 may be configured to execute a molecular structure generation module 20 at which the plurality of training molecular structures 22 are generated. The plurality of training molecular structures 22 may be generated programmatically, as discussed in further detail below.

The one or more processing devices 12 may be further configured to execute a feature matrix generation module 24. Generating the training data set 50 may further include, at the feature matrix generation module 24, computing a respective plurality of training Hamiltonians 26 of the training molecular structures 22. The respective training Hamiltonian 26 of each training molecular structure 22 may be expressed as a four-index tensor G_{u,v,w,x} that encodes electromagnetic interactions of the electrons with the nuclei of the atoms and with each other. The nuclei of the atoms included in the training molecular structure 22 may be approximated as having fixed locations when the training Hamiltonian 26 is generated.

In some examples, generating the training data set 50 may further include, at the feature matrix generation module 24, generating a plurality of training molecular orbital feature matrices 28 based at least in part on the plurality of training Hamiltonians 26. When the one or more processing devices 12 generates a training molecular orbital feature matrix 28, the one or more processing devices 12 may be configured to generate a training Fock matrix 70 and a training composite two-electron integral matrix 72 from the training Hamiltonian 26. The molecular orbital feature matrices 28 may be expressed elementwise as:

$${M}_{i , j} = {h}_{i , j} + {\sum }_{k , l} {D}_{k , l} {G}_{i , j , k , l}$$

In the above equation, M is the molecular orbital feature matrix 28, h is a term of the training Hamiltonian 26, G is a four-index tensor of Hamiltonian parameters, and D is a density matrix. The density matrix D may be computed when Hartree-Fock estimation is performed, as discussed below.

Each of the molecular orbital feature matrices 28 may encode a respective graph that describes the respective training molecular structure 22 and the training Hamiltonian 26 associated with that training molecular structure 22. In the example of FIGS. 1 and 2, the electron energy estimation machine learning model 60 is a graph neural network. Accordingly, the electron energy estimation machine learning model 60 may be trained to receive inputs in the form of attributed graphs G(V, E, X_{V}, X_{E}, X_{G}). In this expression for an attributed graph, V indicates a plurality of vertices, E indicates one or more edges, X_{V} indicates a plurality of vertex attributes, X_{E} indicates one or more edge attributes, and X_{G} indicates one or more global attributes. The elements of each of the molecular orbital feature matrices 28 may be weighted to indicate the vertex attributes X_{V} and the edge attributes X_{E} as well as the topology of the atoms and bonds included in the corresponding training molecular structure 22. Each of the training molecular orbital feature matrices 28 may include a plurality of training vertex inputs 74 including a plurality of on-diagonal elements 74A of the training Fock matrix 70 and a plurality of on-diagonal elements 74B of the training composite two-electron integral matrix 72. In addition, each of the training molecular orbital feature matrices 28 may further include a plurality of training edge inputs 76 including a plurality of off-diagonal elements 76A of the training Fock matrix 70 and a plurality of off-diagonal elements 76B of the training composite two-electron integral matrix 72. The plurality of vertices V, the one or more edges E, the plurality of vertex attributes X_{V}, and the one or more edge attributes X_{E} may be indicated by the elements of the training molecular orbital feature matrix 28 received from the training Fock matrix 70. The plurality of vertices V and the plurality of vertex attributes X_{V} may be indicated by the elements of the training molecular orbital feature matrix 28 located on the main diagonal. The one or more edges E and the one or more edge attributes X_{E} may be indicated by the off-diagonal elements of the training molecular orbital feature matrix 28.

The global attributes X_{G} of the attributed graph may be indicated by the elements of the training molecular orbital feature matrix 28 received from the training composite two-electron integral matrix 72 and may indicate active orbitals of the training molecular structure 22. When no orbitals are active, the elements received from the training composite two-electron integral matrix 72 may equal zero. By generating the plurality of training molecular orbital feature matrices 28, the one or more processing devices 12 may be configured to encode the plurality of training Hamiltonians 26 in a form in which the training Hamiltonians 26 may be processed efficiently.

Returning to FIG. 1, Generating the training data set 50 may further include, at the one or more processing devices 12, computing a plurality of training energy terms 30 associated with the training molecular structures 22 based at least in part on the plurality of training Hamiltonians 26. In examples in which a plurality of training molecular orbital feature matrices 28 are generated, the one or more processing devices 12 may be configured to generate the plurality of training energy terms 30 based at least in part on the plurality of training molecular orbital feature matrices 28. The plurality of training energy terms 30 may be used as training outputs when training the electron energy estimation machine learning model 60, as discussed in further detail below.

As depicted in FIG. 1, the plurality of training energy terms 30 may include a plurality of kinetic energy terms 32, a plurality of nuclear potential energy terms 34, a plurality of electron repulsion energy terms 36, a plurality of exchange energy terms 38, a plurality of dynamical correlation energy terms 40, and a plurality of static correlation energy terms 42. The total electronic energy for a training molecular structure 22 may be given by the sum of the above terms as approximated for that training molecular structure 22. The kinetic energy term 32 for a training molecular structure 22 may indicate the total kinetic energy of the electrons included in that training molecular structure 22. The nuclear potential energy term 34 may indicate potential energy of the electrons resulting from the charges of the nuclei included in the training molecular structure 22. The electron repulsion energy term 36 may indicate potential energy resulting from mean-field electromagnetic repulsion between the electrons. The exchange energy term 38 may be a term that is included to account for the indistinguishability of electrons. The dynamical correlation energy term 40 may be a term that accounts for correlation between movement of the electrons. The static correlation energy term 42 may be a term that accounts for correlation between electron energies due to the shapes of active electron orbitals.

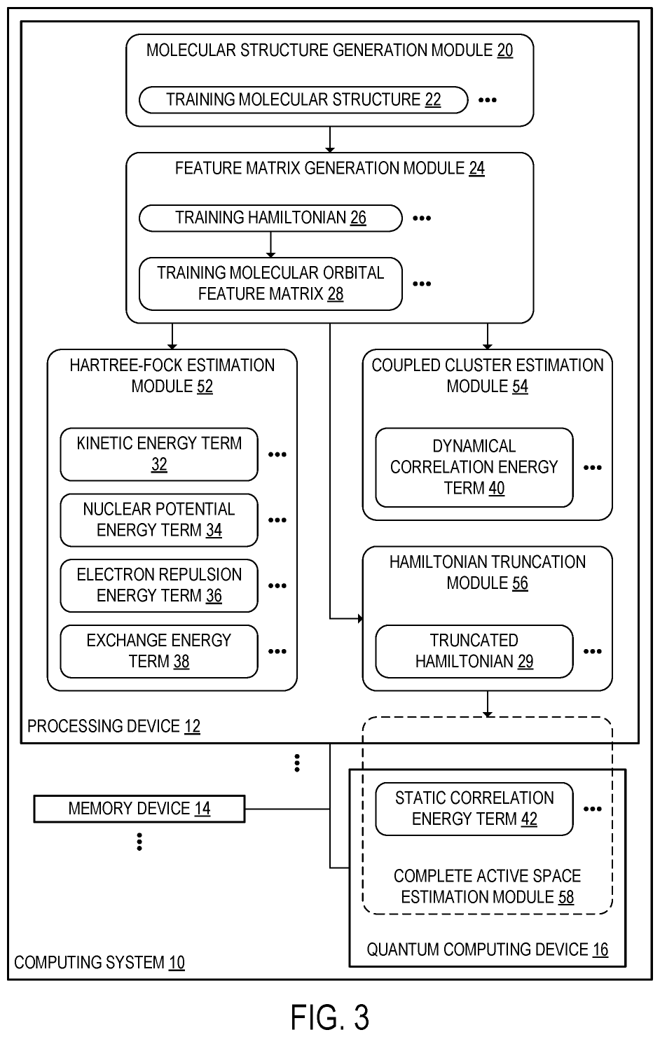

FIG. 3 schematically shows the computing system 10 of FIG. 1 when the one or more processing devices 12 are configured to generate the plurality of training energy terms 30 included in the training data set 50. When generating the plurality of training energy terms 30, the one or more processing devices 12 may be configured to execute a Hartree-Fock estimation module 52 at which the one or more processing devices 12 may be configured to compute respective estimated values of the kinetic energy term 32, the nuclear potential energy term 34, the electron repulsion energy term 36, and the exchange energy term 38 for each of the training Hamiltonians 26. The Hartree-Fock estimation module 52 may be configured to receive the training molecular orbital feature matrix 28 as input. When approximating the above training energy terms 30 at the Hartree-Fock estimation module 52, the one or more processing devices 12 may be configured to approximate the training Hamiltonian 26 as a sum of a plurality of one-electron Fock operators. The one or more processing devices 12 may be further configured to compute an estimated solution to the Schrödinger equation based at least in part on the plurality of one-electron Fock operators to obtain the kinetic energy term 32, the nuclear potential energy term 34, the electron repulsion energy term 36, and the exchange energy term 38. In some examples, the one or more processing devices 12 may be configured to compute a total of the above training energy terms 30 rather than computing the above training energy terms 30 individually.

The one or more processing devices 12 may be further configured to execute a coupled cluster estimation module 54. The coupled cluster estimation module 54 may be configured to receive the training molecular orbital feature matrix 28 as input. At the coupled cluster estimation module 54, the one or more processing devices 12 may be further configured to compute respective dynamical correlation energy terms 40 for a plurality of the training Hamiltonians 26 using coupled cluster estimation. In some examples, the coupled cluster estimation performed at the coupled cluster estimation module 54 may be coupled cluster single-double-triple (CCSD(T)) estimation. In such examples, the training Hamiltonian 26 is approximated as e^{T}, where T is a cluster operator. The cluster operator T is expressed as a sum of a single-excitation term, a double-excitation term, and a triple-excitation term. The parentheses around the T in CCSD(T) indicate that the triple-excitation term is approximated using many-body perturbation theory. In other examples, the one or more processing devices 12 may be configured to use a different coupled cluster estimation technique, such as coupled cluster single-double (CCSD) estimation or coupled cluster single-double-triple (CCSDT) estimation in which the triple term is not computed perturbatively.

For each of a plurality of training Hamiltonians 26, the one or more processing devices 12 may be further configured to generate a respective truncated Hamiltonian 29 for the training molecular structure 22 at a Hamiltonian truncation module 56. As shown in the example of FIG. 3, each truncated Hamiltonian 29 may be a truncated Hamiltonian feature matrix generated at least in part by truncating and sparsifying the training molecular orbital feature matrix 28. The one or more processing devices 12 may, for example, be configured to sparsify the training molecular orbital feature matrix 28 at least in part via element threshold truncation or perturbation-based criteria truncation. Truncating the training molecular orbital feature matrix 28 may generate a truncated Hamiltonian 29 with a reduced number of terms and a reduced norm relative to the training molecular orbital feature matrix 28.

When the training Hamiltonian 26 is sparsified via element threshold truncation, the elements of the truncated Hamiltonian 29 may be computed as follows:

$${G}_{i , j , k , l} ← \{ \begin{matrix}{G}_{i , j , k , l} , & \vert {G}_{i , j , k , l} \vert \geq threshold \\ 0 , & \vert {G}_{i , j , k , l} \vert < threshold\end{matrix}$$

When the training Hamiltonian 26 is sparsified via perturbation-based criteria truncation, a perturbation criterion I may be computed as

$$I = \frac{{\vert {G}_{i , j , k , l} \vert}^{2}}{{ϵ}_{i} + {ϵ}_{j} - {ϵ}_{k} - {ϵ}_{l}}$$

where ϵ is an orbital energy computed during execution of the Hartree-Fock estimation module 52. During perturbation-based criteria truncation, the elements of the truncated Hamiltonian 29 may be computed as follows:

$${G}_{i , j , k , l} ← \{ \begin{matrix}{G}_{i , j , k , l} , & I \geq threshold \\ 0 , & I < threshold\end{matrix}$$

The one or more processing devices 12 may be configured to generate the truncated Hamiltonian 29 such that the truncated Hamiltonian 29 has a same active space as the training Hamiltonian 26. Since the static correlation energy term 42 for a molecule depends upon the active orbitals for that molecule, truncating the training Hamiltonian 26 may result in a truncated Hamiltonian 29 that has the same static correlation energy term 42 as the training Hamiltonian 26.

Subsequently to computing the truncated Hamiltonian 29, the one or more processing devices 12 may be further configured to compute the static correlation energy term 42 based at least in part on the truncated Hamiltonian 29. The static correlation energy term 42 may be computed using complete active space (CAS) estimation at a complete active space estimation module 58. CAS estimation may include computing respective Slater determinants of one or more core orbitals, active orbitals, and/or virtual orbitals. Core orbitals are orbitals occupied by two electrons, active orbitals are orbitals occupied by one electron, and virtual orbitals are orbitals occupied by zero electrons. The wavefunction of the electrons may then be estimated as a linear combination of the Slater determinants. The one or more processing devices 12 may be further configured to compute the static correlation energy term 42 for the truncated Hamiltonian 29 based at least in part on the estimated wavefunction computed using CAS estimation. In some examples, the static correlation energy terms 42 computed for the truncated Hamiltonians 29 may be estimated at least in part via complete-active-space configuration interaction (CAS-CI) estimation.

In some examples, as shown in FIG. 3, the static correlation energy terms 42 for the training molecular structures 22 may be estimated at least in part at the quantum computing device 16. In such examples, when each of the plurality of static correlation energy terms 42 is computed, the quantum computing device 16 may be configured to receive, as input, a four-index tensor G_{u,v,w,x} of Hamiltonian parameters that encode the truncated Hamiltonian 29. The quantum computing device 16 may be further configured to output the static correlation energy term 42 for the truncated Hamiltonian 29 to one or more classical processing devices included in the one or more processing devices 12. Alternatively, the quantum computing device 16 may be configured to output an intermediate value that may be utilized at the the one or more processing devices 12 to compute the static correlation energy term 42.

In other examples, the plurality of static correlation energy terms 42 may be generated at a classical computing device included among the one or more processing devices 12, rather than at a quantum computing device 16. For example, the plurality of static correlation energy terms 42 may be computed at least in part at a specialized hardware accelerator.

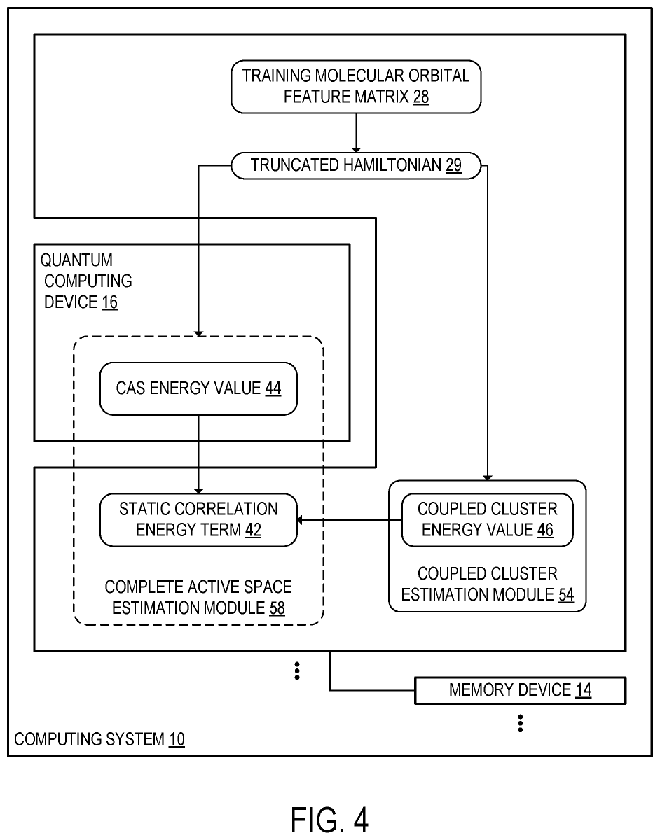

FIG. 4 schematically shows the computation of a static correlation energy term 42 in additional detail. As shown in the example of FIG. 4, for each truncated Hamiltonian 29, the one or more processing devices 12 may be configured to compute the respective static correlation energy term 42 at least in part by computing a CAS energy value 44 and a corresponding coupled cluster energy value 46 for the truncated Hamiltonian 29. The one or more processing devices 12 may be further configured to compute the static correlation energy term 42 as a difference between the CAS energy value 44 and the coupled cluster energy value 46. As shown in the example of FIG. 4, the coupled cluster energy value 46 may be computed at the HF estimation module 52, the CAS energy value 44 may be computed at a portion of the CAS estimation module 58 executed at the quantum computing device 16, and the static correlation energy term 42 may be computed at a portion of the CAS estimation module 58 executed at a classical processing device included among the one or more processing devices 12.

Computing the static correlation energy term 42 as shown in the example of FIG. 4 may allow the one or more processing devices 12 to correct for approximations made when the truncated Hamiltonian 29 is generated from the training Hamiltonian 26. These approximations may lead to inaccuracies in the portion of the CAS energy value 44 corresponding to the sum of the kinetic energy term 32, the nuclear potential energy term 34, the electron repulsion energy term 36, and the exchange energy term 38. Since these portions of the total energy may be estimated accurately using CCSD(T) estimation, the one or more processing devices 12 may be configured to compute the coupled cluster energy value 46 for the truncated Hamiltonian 29 to approximate a total of a kinetic energy term, a nuclear potential energy term, an electron repulsion energy term, an exchange energy term, and a dynamical correlation energy term for the truncated Hamiltonian 29. Since the active space of the training Hamiltonian 26 is preserved when the truncated Hamiltonian 29 is generated, the static correlation energy term 42 may still be accurate despite the truncated Hamiltonian 29 corresponding to an unphysical configuration of electrons. Thus, the static correlation energy term 42 may be approximated accurately by subtracting the coupled cluster energy value 46 from the CAS energy value 44.

The training total electronic energy 62 may be approximated by the following equation:

E_{final}≈E_{HF}+E_{CCSD(T)}^{correlation}+E_{CAS-CI}^{correlation}+E_{CAS-CCSD(T)}^{correlation}

In the above equation, E_{HF} is the sum of the plurality of training energy terms 30 estimated at the Hartree-Fock module 52, E_{CCSD(T)}^{correlation} is the dynamical correlation energy term 40 estimated at the coupled cluster estimation module 54, E_{CAS-CI}^{correlation} is the CAS energy value 44 estimated at the CAS estimation module 58, and E_{CAS-CCSD(T)}^{correlation} is the coupled cluster energy value 46 that is estimated at the coupled cluster estimation module 54 for the truncated Hamiltonian 29. In the above equation, E_{CAS-CCSD(T)}^{correlation} is subtracted from the total on the righthand side to avoid double-counting the dynamical correlation energy term 40.

Returning to FIG. 1, subsequently to computing the plurality of training energy terms 30, the one or more processing devices 12 may be further configured to train the electron energy estimation machine learning model 60 using the plurality of training molecular structures 22 and the plurality of training energy terms 30 included in the training data set 50. The one or more processing devices 12 may be configured to compute a training total electronic energy 62 as a sum of the plurality of training energy terms 30. When training the electron energy estimation machine learning model 60, the one or more processing devices 12 may be configured to perform gradient descent, with the training total electronic energies 62 acting as ground-truth labels for the respective training molecular structures 22 for which they were generated. Thus, the electron energy estimation machine learning model 60 may be trained to predict the total electronic energies of molecular structures that are received as input.

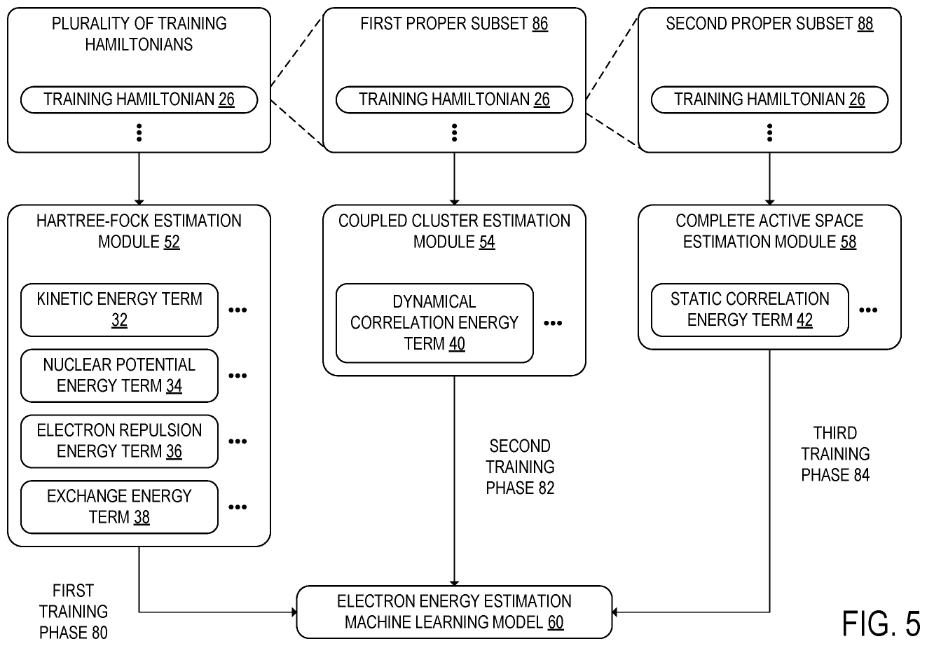

FIG. 5 schematically shows a first training phase 80, a second training phase 82, and a third training phase 84 in which the one or more processing devices 12 may be configured to train the electron energy estimation machine learning model 60. In the first training phase 80, the one or more processing devices 12 may be configured to train the electron energy estimation machine learning model 60 based at least in part on the kinetic energy terms 32, the nuclear potential energy terms 34, the electron repulsion energy terms 36, and the exchange energy terms 38 generated at the HF estimation module 52. In the second training phase 82, the one or more processing devices 12 may be configured to train the electron energy estimation machine learning model 60 based at least in part on the dynamical correlation energy terms 40 generated at the coupled cluster estimation module 54. In the third training phase 84, the one or more processing devices 12 may be configured to train the electron energy estimation machine learning model 60 based at least in part on the static correlation energy terms 42 generated at the CAS estimation model 58. Thus, the one or more processing devices 12 may be configured to perform pre-training during the first training phase 80, perform additional pre-training during the second training phase 82, and perform fine-tuning during the third training phase 84.

As shown in the example of FIG. 5, the one or more processing devices 12 may be configured to use decreasing numbers of training Hamiltonians across the training phases in which the electron energy estimation machine learning model 60 is trained. The plurality of dynamical correlation energy terms 40 may be computed for each training Hamiltonian 26 included in a first proper subset 86 of the plurality of training Hamiltonians 26. In addition, the plurality of static correlation energy terms 42 may be computed for each training Hamiltonian 26 included in a second proper subset 88 of the first proper subset 86. Thus, the one or more processing devices 12 may be configured to generate fewer of the training energy terms 30 that are more computationally expensive to compute. Since the kinetic energy term 32, the nuclear potential energy term 34, the electron repulsion energy term 36, and the exchange energy term 38 typically account for over 95% of the total electronic energy, the dynamical correlation energy term 40 typically accounts for less than 5%, and the static correlation energy term 42 typically accounts for less than 1%, the electron energy estimation machine learning model 60 may achieve high accuracy when predicting the total electronic energy despite the reduced amounts of training data used in the second training phase 82 and the third training phase 84 relative to the first training phase 80.

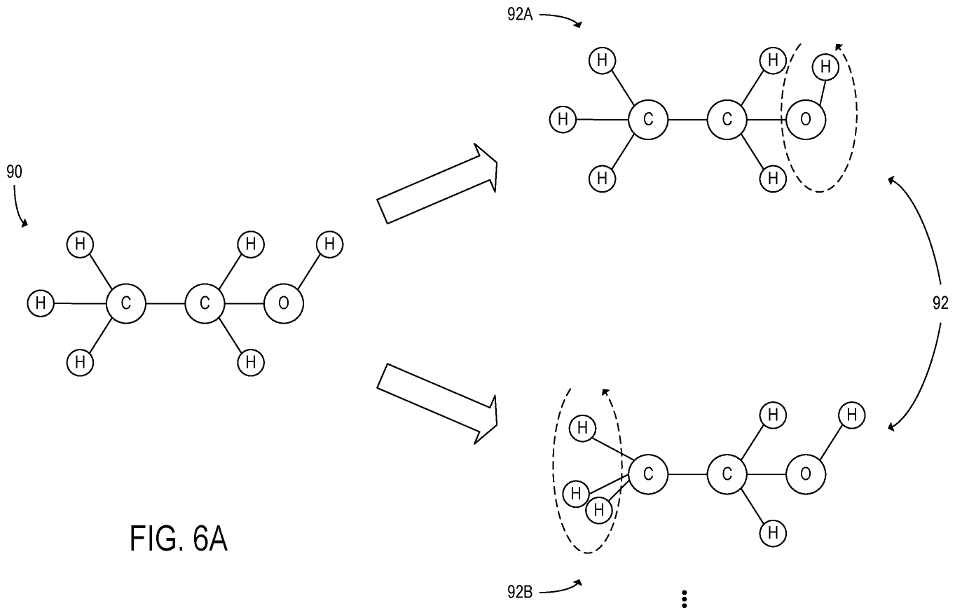

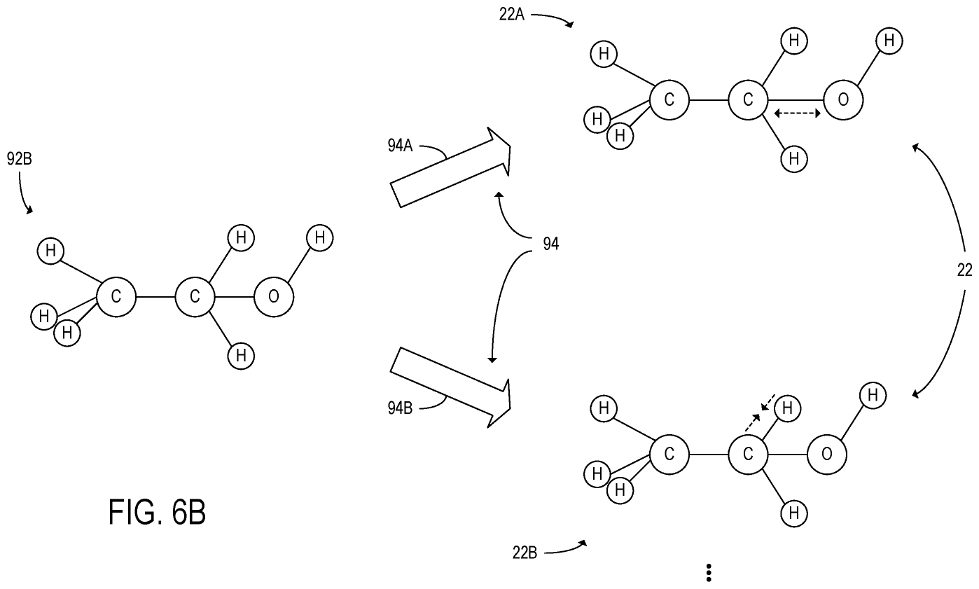

FIGS. 6A-6B show examples in which training molecular structures 22 are generated for inclusion in the training data set 50. As shown in FIG. 6A, the one or more processing devices 12 may be configured to generate the plurality of training molecular structures 22 at least in part by generating a plurality of conformers 92 of one or more stable molecules 90. The conformers 92 are copies of the stable molecule 90 that differ only by rotation of one or more functional groups. In the example of FIG. 6A, a plurality of conformers 92 of ethanol (CH₃CH₂OH) are generated. In a first conformer 92A, the OH group of the ethanol molecule is rotated. In the second conformer 92B, the CH₃ group is rotated. The one or more processing devices 12 may be further configured to generate one or more additional conformers 92 beyond those shown in FIG. 6A.

As shown in FIG. 6B, the one or more processing devices 12 may be further configured to apply a plurality of perturbations 94 to each of the conformers 92 to obtain the plurality of training molecular structures 22. The example of FIG. 6B shows a first perturbation 94A and a second perturbation 94B performed on the second conformer 92B of FIG. 6A. Each of the perturbations 94 includes a modification to a position of at least one atom in the molecule such that the molecule is out of equilibrium. The first perturbation 94A in the example of FIG. 6B is an increase in the distance between the oxygen atom of the ethanol molecule and the carbon atom to which that oxygen atom is bonded. The second perturbation 94B is a decrease in the distance between the central carbon atom of the ethanol molecule and one of the hydrogen atoms to which that central carbon atom is bonded. Thus, the one or more processing devices 12 may generate a first training molecular structure 22A and a second training molecular structure 22B by applying the first perturbation 94A and the second perturbation 94B, respectively, to copies of the second conformer 92B.

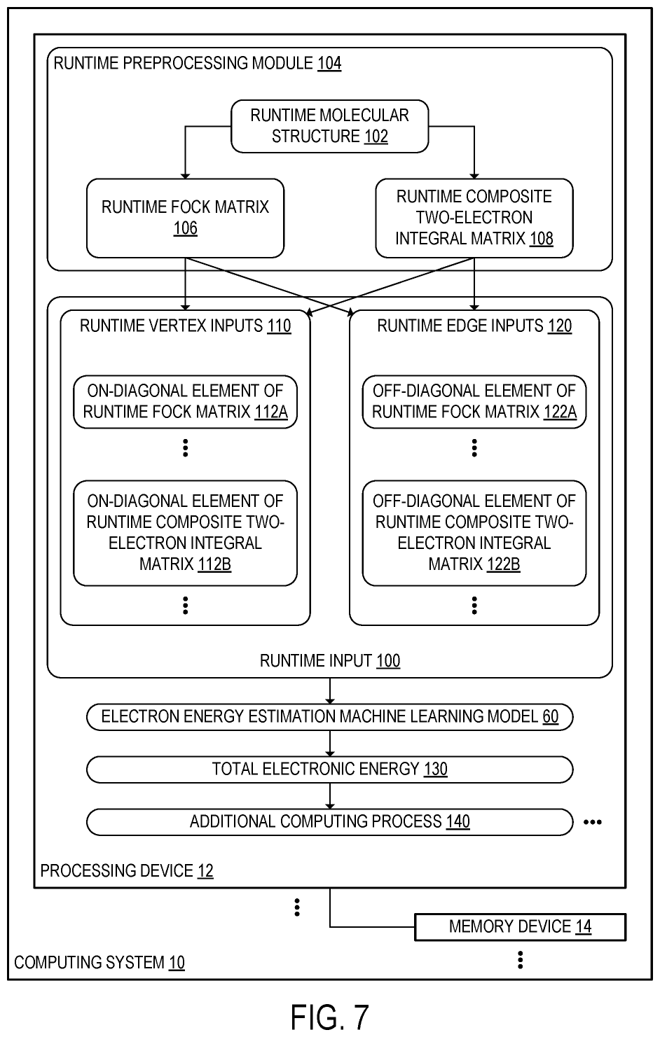

FIG. 7 schematically shows the computing system 10 during runtime when inferencing is performed at the electron energy estimation machine learning model 60. At the electron energy estimation machine learning model 60, the one or more processing devices 12 may be configured to receive a runtime input 100 including a plurality of runtime vertex inputs 110 and a plurality of runtime edge inputs 120 for a runtime molecular structure 102. The plurality of runtime vertex inputs 110 and the plurality of runtime edge inputs 120 may be generated based at least in part on the runtime molecular structure 102 at a runtime preprocessing module 104. The one or more processing devices 12 may, at the runtime preprocessing module 104, be configured to generate a runtime Fock matrix 106 and a runtime composite two-electron integral matrix 108 for the runtime molecular structure 102. The plurality of runtime vertex inputs 110 may include a plurality of on-diagonal elements 112A of the runtime Fock matrix 106 and a plurality of on-diagonal elements 112B of the runtime composite two-electron integral matrix 108. The plurality of runtime vertex inputs 120 may include a plurality of off-diagonal elements 122A of the runtime Fock matrix 106 and a plurality of off-diagonal elements 122B of the runtime composite two-electron integral matrix 108.

At the electron energy estimation machine learning model 60, the one or more processing devices 12 may be further configured to estimate a total electronic energy 130 of the runtime molecular structure 102 based at least in part on the runtime input 100. The one or more processing devices 12 may be further configured to output the total electronic energy 130 to one or more additional computing processes 140. For example, the one or more additional computing processes 140 may include a graphical user interface (GUI) generating module at which the one or more processing devices 12 may be configured to generate a graphical representation of the total electronic energy 130 for output to a user at a GUI displayed on a display device. As another example, the one or more additional computing processes 140 may include a chemical reaction simulation module at which the one or more processing devices 12 may simulate chemical reactions based at least in part on the value of the total electronic energy 130 estimated at the electron energy estimation machine learning model 60.

Although computation of the total electronic energy 130 is discussed above, one or more other properties of a molecule may additionally or alternatively be computed. For example, the one or more processing devices 12 may be configured to compute one or more forces between atoms, a representation of the molecular wavefunction, a dipole moment of the molecule, or one or more electronic transition energies. In such examples, the processor 12 may be configured to compute a plurality of output labels corresponding to a plurality of values of at least one of the above quantities when generating the training data 50 for the electron energy estimation machine learning model 60. Such quantities may be substituted for the training total electronic energies 62 in the training data 50 or may be included in the training data along with corresponding training total electronic energies 62. Thus, during training, the electron energy estimation machine learning model 60 may be trained to predict values of one or more of the above quantities when runtime molecular structures 102 are received as input.

In addition, although the electron energy estimation machine learning model 60 is described above as being configured to generate estimates of total electronic energy 130 for runtime molecular structures 102, the electron energy estimation machine learning model 60 may, in some examples, be trained to estimate total electronic energies 130 of other systems. Thus, in such examples, one or more of the training Hamiltonians 26 may be generated from one or more models other than training molecular structures 22, such as one or more Ising models or Hubbard models.

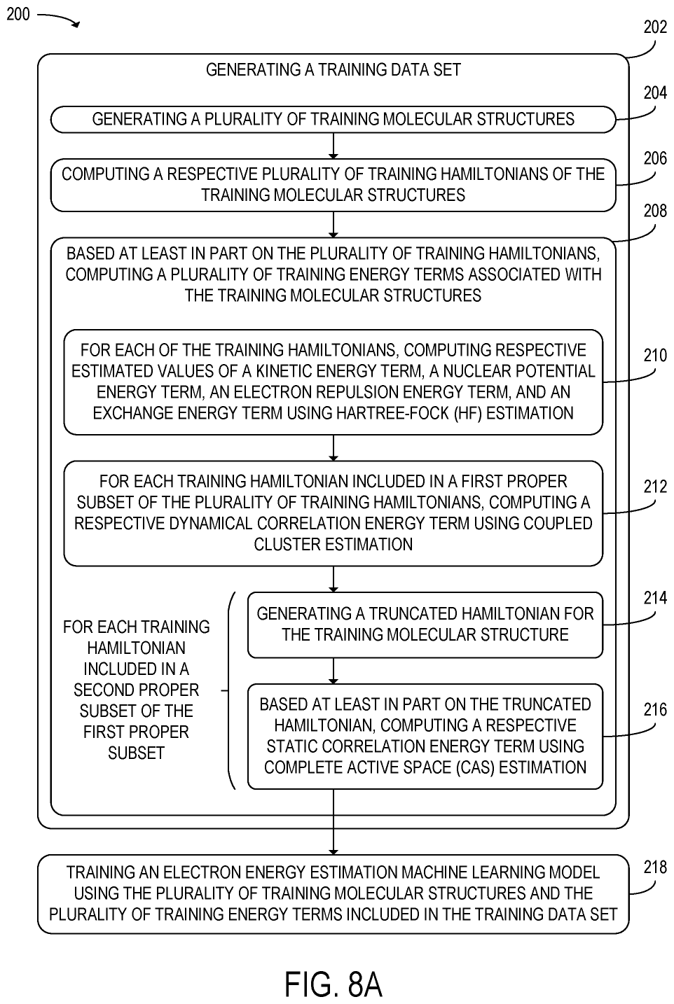

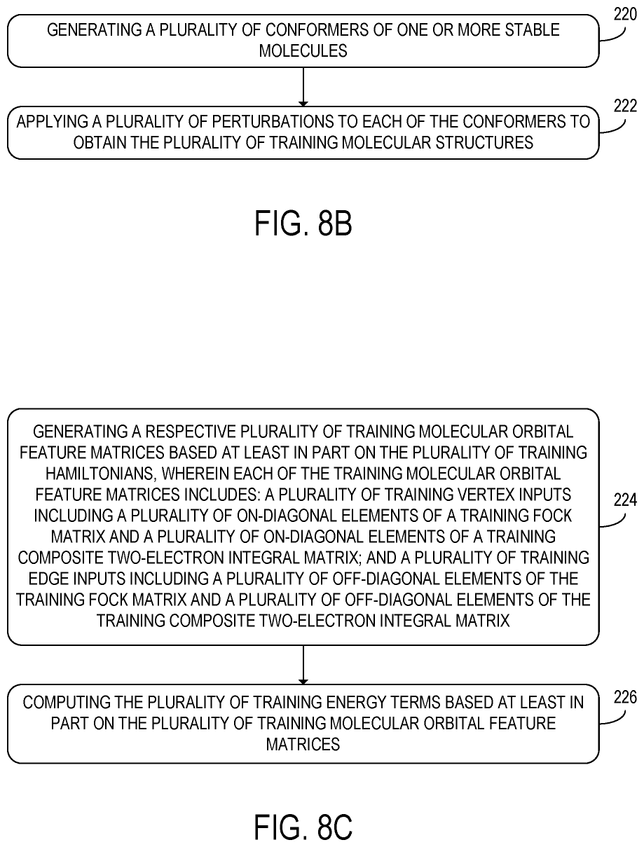

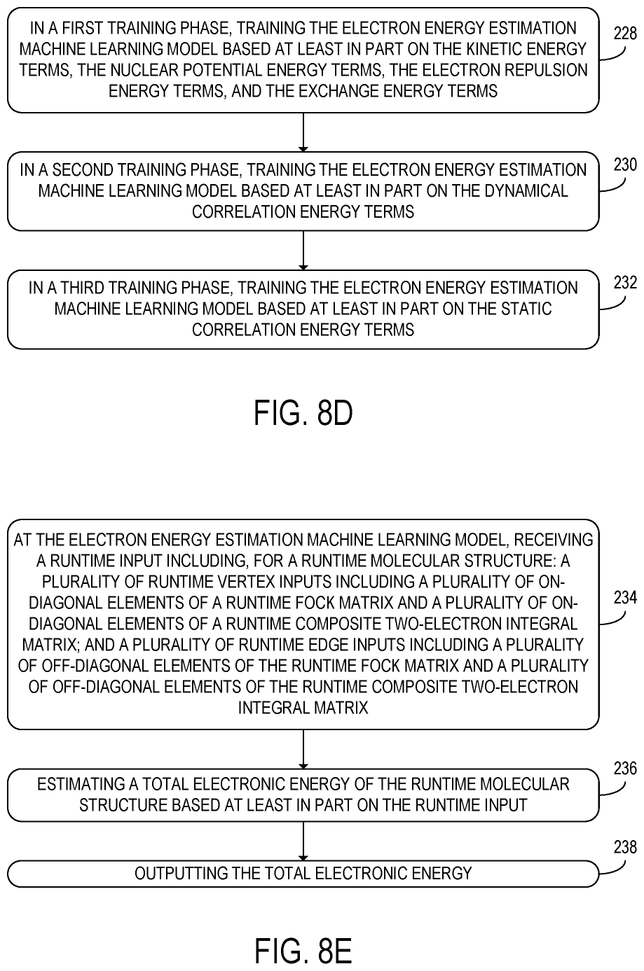

FIG. 8A shows a flowchart of a method 200 for use with a computing system to train an electron energy estimation machine learning model. For example, the method 200 may be performed at the computing system 10 of FIG. 1. At step 202, the method 200 may include generating a training data set with which the electron energy estimation machine learning model may be trained. Step 202 may include, at step 204, generating a plurality of training molecular structures. At step 206, step 202 may further include computing a respective plurality of training Hamiltonians of the training molecular structures.

Generating the training data set at step 202 may further include, at step 208, computing a plurality of training energy terms associated with the training molecular structures based at least in part on the plurality of training Hamiltonians. Computing the plurality of training energy terms at step 208 may include, at step 210, computing respective estimated values of a kinetic energy term, a nuclear potential energy term, an electron repulsion energy term, and an exchange energy term for each of the training Hamiltonians. The estimated values of the kinetic energy term, the nuclear potential energy term, the electron repulsion energy term, and the exchange energy term may be computed using HF estimation.

At step 212, computing the plurality of training energy terms at step 208 may further include computing a respective dynamical correlation energy term for each training Hamiltonian included in a first proper subset of the plurality of training Hamiltonians. The dynamical correlation energy terms may be computed using coupled cluster estimation. For example, the coupled cluster estimation may be CCSD(T) estimation. The dynamical correlation energy terms may be computed for a first proper subset of the training Hamiltonians rather than the complete set of training Hamiltonians due to the higher computational complexity of coupled cluster estimation compared to HF estimation.

Computing the plurality of training energy terms at step 208 may further include steps 214 and 216, which may be performed for each training Hamiltonian included in a second proper subset of the first proper subset. At step 214, the method 200 may further include generating a truncated Hamiltonian for the training molecular structure. At step 216, the method 200 may further include, computing a respective static correlation energy term using CAS estimation based at least in part on the truncated Hamiltonian. For example, the static correlation energy terms may be estimated at least in part via CAS-CI estimation. The static correlation energy terms may be computed for a second proper subset of the first proper subset due to the higher computational complexity of CAS-CI estimation compared to HF estimation and coupled cluster estimation. In some examples, the static correlation energy terms may be estimated at least in part at a quantum computing device.

As step 218, subsequently to generating the training data set at step 202, the method 200 may further include training an electron energy estimation machine learning model using the plurality of training molecular structures and the plurality of training energy terms included in the training data set. The kinetic energy term, the nuclear potential energy term, the electron repulsion energy term, the exchange energy term, the dynamical correlation energy term, and the static correlation energy term for a training molecular structure may sum to the total electronic energy for that training molecular structure. When the electron energy estimation machine learning model is trained, sums of the training energy terms generated for each training molecular structure may be used as ground-truth labels for the training molecular structures. The electron energy estimation machine learning model may be trained via gradient descent. Thus, the electron energy estimation machine learning model may be trained to predict the total electronic energies of molecules from the structures of those molecules.

FIG. 8B shows additional steps of the method 200 that may be performed in some examples when the plurality of training molecular structures are generated. At step 220, the method 200 may further include generating a plurality of conformers of one or more stable molecules. At step 222, the method 200 may further include applying a plurality of perturbations to each of the conformers to obtain the plurality of training molecular structures. Thus, training molecular structures may be generated for non-equilibrium states of stable molecules. Since such non-equilibrium states may occur during chemical reactions, generating the training molecular structures according to steps 220 and 222 may allow the electron energy estimation machine learning model to more accurately predict the total electronic energies those molecules have when chemical reactions occur.

FIG. 8C shows additional steps of the method 200 that may be performed when generating the training data set at step 202 in some examples. In the example of FIG. 8C, the electron energy estimation machine learning model is a graph neural network. At step 224, the method 200 may further include generating a respective plurality of training molecular orbital feature matrices based at least in part on the plurality of training Hamiltonians. Each of the training molecular orbital feature matrices may include a plurality of training vertex inputs and a plurality of training edge inputs. The plurality of training vertex inputs may include a plurality of on-diagonal elements of a training Fock matrix and a plurality of on-diagonal elements of a training composite two-electron integral matrix. The plurality of training edge inputs may include a plurality of off-diagonal elements of the training Fock matrix and a plurality of off-diagonal elements of the training composite two-electron integral matrix. The training vertex inputs may be located on the main diagonal of the training molecular orbital feature matrix and the training edge inputs may be located off the main diagonal of the training molecular orbital feature matrix.

At step 226, the method 200 may further include computing the plurality of training energy terms based at least in part on the plurality of training molecular orbital feature matrices. When the training Hamiltonians are encoded as training molecular orbital feature matrices, the training molecular orbital feature matrices may represent the training Hamiltonians as graph structures that may be used as inputs to a graph neural network. In addition, in examples in which the training Hamiltonians are encoded as training molecular feature orbital matrices, the plurality of truncated Hamiltonians may be generated at step 214 at least in part by truncating and sparsifying the plurality of training molecular orbital feature matrices.

FIG. 8D shows additional steps of the method 200 that may be performed when training the electron energy estimation machine learning model at step 218. At step 228, the method 200 may further include, in a first training phase, training the electron energy estimation machine learning model based at least in part on the kinetic energy terms, the nuclear potential energy terms, the electron repulsion energy terms, and the exchange energy terms. At step 230, the method 200 may further include, in a second training phase, training the electron energy estimation machine learning model based at least in part on the dynamical correlation energy terms. At step 232, the method 200 may further include, in a third training phase, training the electron energy estimation machine learning model based at least in part on the static correlation energy terms. The first training phase and the second training phase may accordingly be first and second pre-training phases, and the third training phase may be a fine-tuning phase.

FIG. 8E shows additional steps of the method 200 that may be performed during runtime in examples in which the electron energy estimation machine learning model is a graph neural network. At step 234, the method 200 may include receiving a runtime input at the electron energy estimation machine learning model. The runtime input may include a plurality of runtime vertex inputs and a plurality of runtime edge inputs. The plurality of runtime vertex inputs may include a plurality of on-diagonal elements of a runtime Fock matrix and a plurality of on-diagonal elements of a runtime composite two-electron integral matrix. The plurality of runtime edge inputs may include a plurality of off-diagonal elements of the runtime Fock matrix and a plurality of off-diagonal elements of the runtime composite two-electron integral matrix. The runtime vertex inputs may be located on the main diagonal of the runtime molecular orbital feature matrix and the runtime edge inputs may be located off the main diagonal of the runtime molecular orbital feature matrix.

At step 236, the method 200 may further include, at the electron energy estimation machine learning model, estimating a total electronic energy of the runtime molecular structure based at least in part on the runtime input. At step 238, the method 200 may further include outputting the total electronic energy. The total electronic energy may be output to an additional computing process such as a GUI generation module or a chemical reaction simulation module.

Using the systems and methods discussed above, an electron energy estimation machine learning model may be trained to predict the total electronic energies of molecules based on those molecules' structures. The static correlation energy terms of training molecular structures may be computed more efficiently using the above systems and methods compared to previous approaches, and an increased number of static correlation energy terms may therefore be utilized when training the electron energy estimation machine learning model. Accordingly, the training techniques discussed above may allow the electron energy estimation machine learning model to predict static correlation terms included in the total electronic energy more accurately than previously existing models. When inferencing is performed at the electron energy estimation machine learning model, the total electronic energies of molecules may be estimated more accurately. The systems and methods discussed above may therefore allow for more accurate simulations of chemical processes.

In some embodiments, the methods and processes described herein may be tied to a computing system of one or more computing devices. In particular, such methods and processes may be implemented as a computer-application program or service, an application-programming interface (API), a library, and/or other computer-program product.

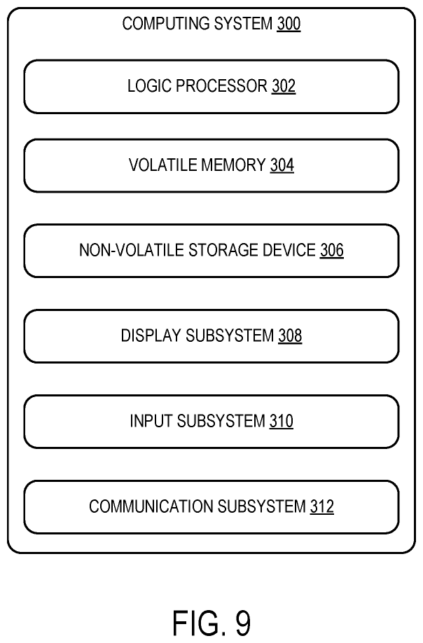

FIG. 9 schematically shows a non-limiting embodiment of a computing system 300 that can enact one or more of the methods and processes described above. Computing system 300 is shown in simplified form. Computing system 300 may embody the computing system 10 described above and illustrated in FIG. 1. Components of the computing system 300 may be instantiated in one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 300 includes a logic processor 302 volatile memory 304, and a non-volatile storage device 306. Computing system 300 may optionally include a display sub system 308, input sub system 310, communication sub system 312, and/or other components not shown in FIG. 9.

Logic processor 302 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 302 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Volatile memory 304 may include physical devices that include random access memory. Volatile memory 304 is typically utilized by logic processor 302 to temporarily store information during processing of software instructions. It will be appreciated that volatile memory 304 typically does not continue to store instructions when power is cut to the volatile memory 304.

Non-volatile storage device 306 includes one or more physical devices configured to hold instructions executable by the logic processors to implement the methods and processes described herein. When such methods and processes are implemented, the state of non-volatile storage device 306 may be transformed—e.g., to hold different data.

Non-volatile storage device 306 may include physical devices that are removable and/or built-in. Non-volatile storage device 306 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 306 may include nonvolatile, dynamic, static, read/write, read-only, sequential-access, location-addressable, file-addressable, and/or content-addressable devices. It will be appreciated that non-volatile storage device 306 is configured to hold instructions even when power is cut to the non-volatile storage device 306.

Aspects of logic processor 302, volatile memory 304, and non-volatile storage device 306 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 300 typically implemented in software by a processor to perform a particular function using portions of volatile memory, which function involves transformative processing that specially configures the processor to perform the function. Thus, a module, program, or engine may be instantiated via logic processor 302 executing instructions held by non-volatile storage device 306, using portions of volatile memory 304. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 308 may be used to present a visual representation of data held by non-volatile storage device 306. The visual representation may take the form of a graphical user interface (GUI). As the herein described methods and processes change the data held by the non-volatile storage device, and thus transform the state of the non-volatile storage device, the state of display subsystem 308 may likewise be transformed to visually represent changes in the underlying data. Display subsystem 308 may include one or more display devices utilizing virtually any type of technology. Such display devices may be combined with logic processor 302, volatile memory 304, and/or non-volatile storage device 306 in a shared enclosure, or such display devices may be peripheral display devices.

When included, input subsystem 310 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 312 may be configured to communicatively couple various computing devices described herein with each other, and with other devices. Communication subsystem 312 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 300 to send and/or receive messages to and/or from other devices via a network such as the Internet.

The following paragraphs discuss several aspects of the present disclosure. According to one aspect of the present disclosure, a computing system is provided, including one or more processing devices configured to generate a training data set. The one or more processing devices may be configured to generate the training data set at least in part by generating a plurality of training molecular structures. Generating the training data set may further include computing a respective plurality of training Hamiltonians of the training molecular structures. Generating the training data set may further include, based at least in part on the plurality of training Hamiltonians, computing a plurality of training energy terms associated with the training molecular structures. Computing the plurality of training energy terms may include, for each of the training Hamiltonians, computing respective estimated values of a kinetic energy term, a nuclear potential energy term, an electron repulsion energy term, and an exchange energy term using Hartree-Fock (HF) estimation. For each training Hamiltonian included in a first proper subset of the plurality of training Hamiltonians, computing the plurality of training energy terms may further include computing a respective dynamical correlation energy term using coupled cluster estimation. For each training Hamiltonian included in a second proper subset of the first proper subset, computing the plurality of training energy terms may further include generating a truncated Hamiltonian for the training molecular structure, and based at least in part on the truncated Hamiltonian, computing a respective static correlation energy term using complete active space (CAS) estimation. The processor may be further configured to train an electron energy estimation machine learning model using the plurality of training molecular structures and the plurality of training energy terms included in the training data set.

According to this aspect, the electron energy estimation machine learning model may be a graph neural network.

According to this aspect, the one or more processing devices may be further configured to, when computing the plurality of training energy terms, generate a respective plurality of training molecular orbital feature matrices based at least in part on the plurality of training Hamiltonians. Each of the training molecular orbital feature matrices may include a plurality of training vertex inputs including a plurality of on-diagonal elements of a training Fock matrix and a plurality of on-diagonal elements of a training composite two-electron integral matrix. Each of the training molecular orbital feature matrices may further include a plurality of training edge inputs including a plurality of off-diagonal elements of the training Fock matrix and a plurality of off-diagonal elements of the training composite two-electron integral matrix. The processor may be further configured to compute the plurality of training energy terms based at least in part on the plurality of training molecular orbital feature matrices.

According to this aspect, during runtime, the one or more processing devices are configured to, at the electron energy estimation machine learning model, receive a runtime input. The runtime input may include, for a runtime molecular structure, a plurality of runtime vertex inputs including a plurality of on-diagonal elements of a runtime Fock matrix and a plurality of on-diagonal elements of a runtime composite two-electron integral matrix. The runtime input may further include a plurality of runtime edge inputs including a plurality of off-diagonal elements of the runtime Fock matrix and a plurality of off-diagonal elements of the runtime composite two-electron integral matrix. The one or more processing devices may be further configured to estimate a total electronic energy of the runtime molecular structure based at least in part on the runtime input. The one or more processing devices may be further configured to output the total electronic energy.

According to this aspect, the one or more processing devices may be configured to generate the plurality of truncated Hamiltonians at least in part by truncating and sparsifying the plurality of training molecular orbital feature matrices.

According to this aspect, the static correlation energy terms may be estimated at least in part at a quantum computing device.

According to this aspect, the static correlation energy terms are estimated at least in part via complete-active-space configuration interaction (CAS-CI) estimation.

According to this aspect, the coupled cluster estimation may be coupled cluster single-double-triple (CCSD(T)) estimation.

According to this aspect, for each truncated Hamiltonian, the one or more processing devices are configured to compute the respective static correlation energy term at least in part by computing a CAS energy value and a corresponding coupled cluster energy value for the truncated Hamiltonian. The static correlation energy term may be computed as a difference between the CAS energy value and the coupled cluster energy value.

According to this aspect, when training the electron energy estimation machine learning model, the one or more processing devices may be configured to, in a first training phase, train the electron energy estimation machine learning model based at least in part on the kinetic energy terms, the nuclear potential energy terms, the electron repulsion energy terms, and the exchange energy terms. The one or more processing devices may be further configured to, in a second training phase, train the electron energy estimation machine learning model based at least in part on the dynamical correlation energy terms. The one or more processing devices may be further configured to, in a third training phase, train the electron energy estimation machine learning model based at least in part on the static correlation energy terms.

According to this aspect, the one or more processing devices are configured to generate the plurality of training molecular structures at least in part by generating a plurality of conformers of one or more stable molecules. The one or more processing devices may be further configured to apply a plurality of perturbations to each of the conformers to obtain the plurality of training molecular structures.

According to another aspect of the present disclosure, a method for use with a computing system is provided. The method may include generating a training data set at least in part by generating a plurality of training molecular structures. Generating the training data set may further include computing a respective plurality of training Hamiltonians of the training molecular structures. Generating the training data set may further include, based at least in part on the plurality of training Hamiltonians, computing a plurality of training energy terms associated with the training molecular structures. Computing the plurality of training energy terms may include, for each of the training Hamiltonians, computing respective estimated values of a kinetic energy term, a nuclear potential energy term, an electron repulsion energy term, and an exchange energy term using Hartree-Fock (HF) estimation. Computing the plurality of training energy terms may further include, for each training Hamiltonian included in a first proper subset of the plurality of training Hamiltonians, computing a respective dynamical correlation energy term using coupled cluster estimation. Computing the plurality of training energy terms may further include, for each training Hamiltonian included in a second proper subset of the first proper subset, generating a truncated Hamiltonian for the training molecular structure, and based at least in part on the truncated Hamiltonian, computing a respective static correlation energy term using complete active space (CAS) estimation. The method may further include training an electron energy estimation machine learning model using the plurality of training molecular structures and the plurality of training energy terms included in the training data set.

According to this aspect, the electron energy estimation machine learning model may be a graph neural network.

According to this aspect, the method may further include generating a respective plurality of training molecular orbital feature matrices based at least in part on the plurality of training Hamiltonians. Each of the training molecular orbital feature matrices may include a plurality of training vertex inputs including a plurality of on-diagonal elements of a training Fock matrix and a plurality of on-diagonal elements of a training composite two-electron integral matrix. Each of the training molecular orbital feature matrices may further include a plurality of training edge inputs including a plurality of off-diagonal elements of the training Fock matrix and a plurality of off-diagonal elements of the training composite two-electron integral matrix. The method may further include computing the plurality of training energy terms based at least in part on the plurality of training molecular orbital feature matrices.

According to this aspect, the method may further include, during runtime, receiving a runtime input at the electron energy estimation machine learning model. The runtime input may include, for a runtime molecular structure, a plurality of runtime vertex inputs including a plurality of on-diagonal elements of a runtime Fock matrix and a plurality of on-diagonal elements of a runtime composite two-electron integral matrix. The runtime input may further include a plurality of runtime edge inputs including a plurality of off-diagonal elements of the runtime Fock matrix and a plurality of off-diagonal elements of the runtime composite two-electron integral matrix. The method may further include estimating a total electronic energy of the runtime molecular structure based at least in part on the runtime input. The method may further include outputting the total electronic energy.

According to this aspect, the static correlation energy terms may be estimated at least in part at a quantum computing device.

According to this aspect, the static correlation energy terms may be estimated at least in part via complete-active-space configuration interaction (CAS-CI) estimation.

According to this aspect, the coupled cluster estimation may be coupled cluster single-double-triple (CCSD(T)) estimation.

According to this aspect, training the electron energy estimation machine learning model may include, in a first training phase, training the electron energy estimation machine learning model based at least in part on the kinetic energy terms, the nuclear potential energy terms, the electron repulsion energy terms, and the exchange energy terms. Training the electron energy estimation machine learning model may further include, in a second training phase, training the electron energy estimation machine learning model based at least in part on the dynamical correlation energy terms. Training the electron energy estimation machine learning model may further include, in a third training phase, training the electron energy estimation machine learning model based at least in part on the static correlation energy terms.

According to another aspect of the present disclosure, a computing system is provided, including one or more processing devices configured to generate a training data set. Generating the training data set may include generating a plurality of training molecular structures. Generating the training data set may further include computing a respective plurality of training Hamiltonians of the training molecular structures. Generating the training data set may further include, based at least in part on the plurality of training Hamiltonians, computing a plurality of training energy terms associated with the training molecular structures. Computing the plurality of training energy terms may include, for each of the training Hamiltonians, computing respective estimated values of a kinetic energy term, a nuclear potential energy term, an electron repulsion energy term, and an exchange energy term. Computing the plurality of training energy terms may further include, for each training Hamiltonian included in a first proper subset of the plurality of training Hamiltonians, computing a respective dynamical correlation energy term. Computing the plurality of training energy terms may further include, for each training Hamiltonian included in a second proper subset of the first proper subset, generating a truncated Hamiltonian for the training molecular structure, and based at least in part on the truncated Hamiltonian, computing a respective static correlation energy term. Using the plurality of training molecular structures and the plurality of training energy terms included in the training data set, the processor may be further configured to train an electron energy estimation machine learning model. Training the electron energy estimation machine learning model may include, in a first training phase, training the electron energy estimation machine learning model based at least in part on the kinetic energy terms, the nuclear potential energy terms, the electron repulsion energy terms, and the exchange energy terms. Training the electron energy estimation machine learning model may further include, in a second training phase, training the electron energy estimation machine learning model based at least in part on the dynamical correlation energy terms. Training the electron energy estimation machine learning model may further include, in a third training phase, training the electron energy estimation machine learning model based at least in part on the static correlation energy terms.

“And/or” as used herein is defined as the inclusive or ∨, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system comprising:
one or more processing devices configured to:
generate a training data set at least in part by:
generating a plurality of training molecular structures;
computing a respective plurality of training Hamiltonians of the training molecular structures;
based at least in part on the plurality of training Hamiltonians, computing a plurality of training energy terms associated with the training molecular structures, wherein computing the plurality of training energy terms includes:
for each of the training Hamiltonians, computing respective estimated values of a kinetic energy term, a nuclear potential energy term, an electron repulsion energy term, and an exchange energy term using Hartree-Fock (HF) estimation;
for each training Hamiltonian included in a first proper subset of the plurality of training Hamiltonians, computing a respective dynamical correlation energy term using coupled cluster estimation; and
for each training Hamiltonian included in a second proper subset of the first proper subset:
generating a truncated Hamiltonian for the training molecular structure; and
based at least in part on the truncated Hamiltonian, computing a respective static correlation energy term using complete active space (CAS) estimation; and
train an electron energy estimation machine learning model using the plurality of training molecular structures and the plurality of training energy terms included in the training data set.

2. The computing system of claim 1, wherein the electron energy estimation machine learning model is a graph neural network.

3. The computing system of claim 2, wherein the one or more processing devices are further configured to, when computing the plurality of training energy terms:
generate a respective plurality of training molecular orbital feature matrices based at least in part on the plurality of training Hamiltonians, wherein each of the training molecular orbital feature matrices includes:
a plurality of training vertex inputs including a plurality of on-diagonal elements of a training Fock matrix and a plurality of on-diagonal elements of a training composite two-electron integral matrix; and
a plurality of training edge inputs including a plurality of off-diagonal elements of the training Fock matrix and a plurality of off-diagonal elements of the training composite two-electron integral matrix; and
compute the plurality of training energy terms based at least in part on the plurality of training molecular orbital feature matrices.

4. The computing system of claim 3, wherein, during runtime, the one or more processing devices are configured to:
at the electron energy estimation machine learning model, receive a runtime input including, for a runtime molecular structure:
a plurality of runtime vertex inputs including a plurality of on-diagonal elements of a runtime Fock matrix and a plurality of on-diagonal elements of a runtime composite two-electron integral matrix; and
a plurality of runtime edge inputs including a plurality of off-diagonal elements of the runtime Fock matrix and a plurality of off-diagonal elements of the runtime composite two-electron integral matrix;
estimate a total electronic energy of the runtime molecular structure based at least in part on the runtime input; and
output the total electronic energy.

5. The computing system of claim 3, wherein the one or more processing devices are configured to generate the plurality of truncated Hamiltonians at least in part by truncating and sparsifying the plurality of training molecular orbital feature matrices.

6. The computing system of claim 1, wherein the static correlation energy terms are estimated at least in part at a quantum computing device.

7. The computing system of claim 1, wherein the static correlation energy terms are estimated at least in part via complete-active-space configuration interaction (CAS-CI) estimation.

8. The computing system of claim 1, wherein the coupled cluster estimation is coupled cluster single-double-triple (CCSD(T)) estimation.

9. The computing system of claim 1, wherein, for each truncated Hamiltonian, the one or more processing devices are configured to compute the respective static correlation energy term at least in part by:
computing a CAS energy value and a corresponding coupled cluster energy value for the truncated Hamiltonian; and
computing the static correlation energy term as a difference between the CAS energy value and the coupled cluster energy value.

10. The computing system of claim 1, wherein, when training the electron energy estimation machine learning model, the one or more processing devices are configured to:
in a first training phase, train the electron energy estimation machine learning model based at least in part on the kinetic energy terms, the nuclear potential energy terms, the electron repulsion energy terms, and the exchange energy terms;
in a second training phase, train the electron energy estimation machine learning model based at least in part on the dynamical correlation energy terms; and
in a third training phase, train the electron energy estimation machine learning model based at least in part on the static correlation energy terms.

11. The computing system of claim 1, wherein the one or more processing devices are configured to generate the plurality of training molecular structures at least in part by:
generating a plurality of conformers of one or more stable molecules; and
applying a plurality of perturbations to each of the conformers to obtain the plurality of training molecular structures.

12. A method for use with a computing system, the method comprising:
generating a training data set at least in part by:
generating a plurality of training molecular structures;
computing a respective plurality of training Hamiltonians of the training molecular structures;
based at least in part on the plurality of training Hamiltonians, computing a plurality of training energy terms associated with the training molecular structures, wherein computing the plurality of training energy terms includes:
for each of the training Hamiltonians, computing respective estimated values of a kinetic energy term, a nuclear potential energy term, an electron repulsion energy term, and an exchange energy term using Hartree-Fock (HF) estimation;
for each training Hamiltonian included in a first proper subset of the plurality of training Hamiltonians, computing a respective dynamical correlation energy term using coupled cluster estimation; and
for each training Hamiltonian included in a second proper subset of the first proper subset:
generating a truncated Hamiltonian for the training molecular structure; and
based at least in part on the truncated Hamiltonian, computing a respective static correlation energy term using complete active space (CAS) estimation; and
training an electron energy estimation machine learning model using the plurality of training molecular structures and the plurality of training energy terms included in the training data set.

13. The method of claim 12, wherein the electron energy estimation machine learning model is a graph neural network.

14. The method of claim 13, further comprising:
generating a respective plurality of training molecular orbital feature matrices based at least in part on the plurality of training Hamiltonians, wherein each of the training molecular orbital feature matrices includes:
a plurality of training vertex inputs including a plurality of on-diagonal elements of a training Fock matrix and a plurality of on-diagonal elements of a training composite two-electron integral matrix; and
a plurality of training edge inputs including a plurality of off-diagonal elements of the training Fock matrix and a plurality of off-diagonal elements of the training composite two-electron integral matrix; and
computing the plurality of training energy terms based at least in part on the plurality of training molecular orbital feature matrices.

15. The method of claim 13, further comprising, during runtime:
at the electron energy estimation machine learning model, receiving a runtime input including, for a runtime molecular structure:
a plurality of runtime vertex inputs including a plurality of on-diagonal elements of a runtime Fock matrix and a plurality of on-diagonal elements of a runtime composite two-electron integral matrix; and
a plurality of runtime edge inputs including a plurality of off-diagonal elements of the runtime Fock matrix and a plurality of off-diagonal elements of the runtime composite two-electron integral matrix;
estimating a total electronic energy of the runtime molecular structure based at least in part on the runtime input; and
outputting the total electronic energy.

16. The method of claim 12, wherein the static correlation energy terms are estimated at least in part at a quantum computing device.

17. The method of claim 12, wherein the static correlation energy terms are estimated at least in part via complete-active-space configuration interaction (CAS-CI) estimation.

18. The method of claim 12, wherein the coupled cluster estimation is coupled cluster single-double-triple (CCSD(T)) estimation.

19. The method of claim 12, wherein training the electron energy estimation machine learning model includes:
in a first training phase, training the electron energy estimation machine learning model based at least in part on the kinetic energy terms, the nuclear potential energy terms, the electron repulsion energy terms, and the exchange energy terms;
in a second training phase, training the electron energy estimation machine learning model based at least in part on the dynamical correlation energy terms; and
in a third training phase, training the electron energy estimation machine learning model based at least in part on the static correlation energy terms.

20. A computing system comprising:
one or more processing devices configured to:
generate a training data set at least in part by:
generating a plurality of training molecular structures;
computing a respective plurality of training Hamiltonians of the training molecular structures;
based at least in part on the plurality of training Hamiltonians, computing a plurality of training energy terms associated with the training molecular structures, wherein computing the plurality of training energy terms includes:
for each of the training Hamiltonians, computing respective estimated values of a kinetic energy term, a nuclear potential energy term, an electron repulsion energy term, and an exchange energy term;
for each training Hamiltonian included in a first proper subset of the plurality of training Hamiltonians, computing a respective dynamical correlation energy term; and
for each training Hamiltonian included in a second proper subset of the first proper subset:
generating a truncated Hamiltonian for the training molecular structure; and
based at least in part on the truncated Hamiltonian, computing a respective static correlation energy term; and
using the plurality of training molecular structures and the plurality of training energy terms included in the training data set, train an electron energy estimation machine learning model at least in part by:
in a first training phase, training the electron energy estimation machine learning model based at least in part on the kinetic energy terms, the nuclear potential energy terms, the electron repulsion energy terms, and the exchange energy terms;
in a second training phase, training the electron energy estimation machine learning model based at least in part on the dynamical correlation energy terms; and
in a third training phase, training the electron energy estimation machine learning model based at least in part on the static correlation energy terms.
